(19) United States
(12) Patent Application Publication
(10) Pub. No.: US 2009/0234388 A1
(43) Pub. Date: Sep. 17, 2009

(54) Spinal Stabilization Connecting Element and System

(75) Inventors: Christopher M Patterson, Olive Branch, MS (US); Hai H Trieu, Cordova, TN (US); Dimitri Protopsaltis, Memphis, TN (US); Jeff R Justis, Collierville, TN (US)

Correspondence Address: MEDTRONIC;Attn: Noreen Johnson - IP Legal Department, 2600 Sofamor Danek Drive, MEMPHIS, TN, 38132, US

(73) Assignee: WARSAW ORTHOPEDIC, INC., Warsaw, IN (US)

(21) Appl. No.: 12/049,322

(22) Filed: Mar. 15, 2008

(51) Int. Cl. A61B 17/58

(52) U.S. Cl. 606/246; 606/278

(57) ABSTRACT

An elongated connecting element for use in a spinal stabilization system comprises a first section, a second section, a first elastomer disposed within the first section, and a second elastomer disposed between the first section and the second section. One of the first elastomer and the second elastomer resists movement of the first section and the second section toward each other and the other of the first elastomer and the second elastomer resists movement of the first section and the second section away from each other.

### BACKGROUND

Elongated connecting elements, such as rods, plates, tethers, wires, cables, and other devices have been implanted along the spinal column and connected between two or more anchors engaged between one or more spinal motion segments. Such connecting elements can provide a rigid construct that resists movement of the spinal motion segment in response to spinal loading or movement of the spinal motion segment by the patient. Other connecting elements can resist loading or movement of the spinal motion segment that creates a tension force on the connecting element; however, the connecting element collapses in response to any compression loading and provides little or no resistance in response to such forces or movement. Still other connecting elements are flexible to permit at least limited spinal motion while providing resistance to loading and motion of the spinal motion segment in one of compression and tension.

### SUMMARY

In one embodiment of the present disclosure, an elongated connecting element for use in a spinal stabilization system comprises a first section, a second section, a first elastomer disposed within the first section, and a second elastomer disposed between the first section and the second section. One of the first elastomer and the second elastomer resists movement of the first section and the second section toward each other and the other of the first elastomer and the second elastomer resists movement of the first section and the second section away from each other.

In another embodiment of the present disclosure, an elongated connecting element is used in a spinal stabilization system. The connecting element comprises first and second end anchors and an elastomeric bumper portion engaged between the first and second end anchors. The elastomeric bumper includes an outer radial surface. Movement of the first and second end anchors toward each other presses the outer radial surface of the bumper radially outward and movement of the first and second end anchors away from each other presses the outer radial surface radially inward.

In another embodiment of the present disclosure, an elongated connecting element is used in a spinal stabilization system. The connecting element comprises a first end anchor comprising a first elongated cylindrical section and an internal bore extending at least partially through the elongated cylindrical section. The connecting element further comprises a second anchor comprising a second elongated cylindrical section and a rod portion extending away from the second elongated cylindrical section. The connecting element further comprises a bumper between the first and second elongated cylindrical sections. The rod portion is sized to extend through the bumper and into the internal bore of the first end anchor.

In another embodiment of the present disclosure, a spinal stabilization system comprises first and second bone connecting assemblies, a flexible elongated connecting element extending between the first and second bone connecting assemblies, and an adjustable sleeve extending over at least a portion of the connecting element.

In another embodiment of the present disclosure, a method of stabilizing a spinal joint comprises inserting a first connecting assembly into a first vertebra and inserting a second connecting assembly into a second vertebra. The method further comprises extending an elongated connecting element between the first and second connecting assemblies and extending an adjustable sleeve over the elongated connecting element. The adjustable sleeve includes a first sleeve portion movably connected to the second sleeve portion. The method further comprises actuating a drive system to adjust a height of the adjustable sleeve by moving the first sleeve portion with respect to the second sleeve portion.

These and other aspects, forms, objects, features, and benefits of the present invention will become apparent from the following detailed drawings and description.

### BRIEF DESCRIPTION OF THE DRAWINGS

In the accompanying drawings, which are incorporated in and constitute a part of the specification, embodiments of the invention are illustrated, which, together with a general description of the invention given above, and the detailed description given below, serve to exemplify the embodiments of this invention.

FIG. 1 is a perspective view of a vertebral joint with a spinal stabilization system according to one embodiment.

FIG. 2A is a cross-sectional view of a spinal device according to one embodiment of the present disclosure.

FIG. 2B is a perspective view of a spinal device according to one embodiment of the present disclosure.

FIG. 3 is a cross-sectional view of a spinal device showing a dampening assembly according to one embodiment of the present disclosure.

FIG. 4 is an exploded view of a spinal device according to one embodiment of the present disclosure.

FIG. 5 is a partial cross-sectional view of a spinal stabilization system according to one embodiment of the present disclosure.

FIG. 6 is a cross-sectional view of a spinal device according to another embodiment of the present disclosure.

FIG. 7 is another cross-sectional view of the embodiment of FIG. 6.

FIG. 7a is a cross-sectional view according to another embodiment of the present disclosure.

FIG. 8 is cross-sectional view of a spinal device according to an embodiment of the present disclosure illustrating resorbable components.

FIG. 9 is cross-sectional view of a spinal device according to another embodiment of the present disclosure illustrating resorbable components.

FIG. 10 is a view of a spinal device according to another embodiment of the present disclosure.

FIG. 11 is a cross-sectional view of a spinal stabilization system according to another embodiment of the present disclosure.

FIG. 12 is a view of spinal device according to another embodiment of the present disclosure.

FIG. 13 is a partial cross-sectional view of the spinal device of FIG. 12.

FIG. 14 is another partial cross-sectional view of the spinal device of FIG. 12.

FIGS. 15-22 are partial cross-sectional views of spinal device according to other embodiments of the present disclosure.

FIG. 23 is a cross-sectional view of a spinal device according to another embodiment of the present disclosure.

FIG. 24 is another cross-sectional view of the spinal device of FIG. 23.

FIGS. 25-26 are cross-sectional views of spinal devices according to other embodiments of the present disclosure.

FIG. 27 is a side view of a spinal stabilization system according to another embodiment of the present disclosure.

FIGS. 28-29 are a partial cross sectional views of different spinal stabilization systems according to other embodiments of the present disclosure.

### DETAILED DESCRIPTION

The present disclosure relates generally to the field of orthopedic surgery, and more particularly to systems and methods for stabilizing a spinal joint or spinal motion segment. For the purposes of promoting an understanding of the principles of the invention, reference will now be made to embodiments or examples illustrated in the drawings, and specific language will be used to describe these examples. It will nevertheless be understood that no limitation of the scope of the invention is thereby intended. Any alteration and further modifications in the described embodiments, and any further applications of the principles of the invention as described herein, are contemplated as would normally occur to one skilled in the art to which the disclosure relates.

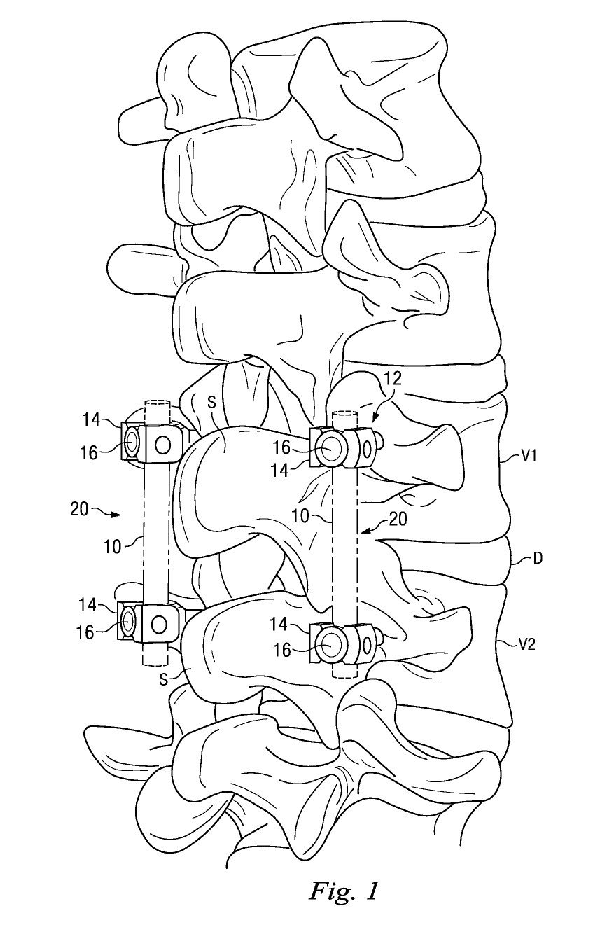

Referring first to FIG. 1, a spinal stabilization system is indicated generally by the numeral 20. Various specific embodiments of the spinal stabilization system will be described in detail below. FIG. 1 shows a perspective view of first and second spinal stabilization systems 20 in which elongated connecting elements or spinal devices 10 are attached to vertebral members V1 and V2. The spinal devices 10 are schematically depicted to indicate that that the spinal devices may be arranged in variety of different shapes and configurations as will be disclosed in the embodiments that follow. The present discussion describes the invention as implanted between two adjacent vertebrae for ease of description, but the invention is not limited to implantation between two adjacent vertebrae. A vertebral disc D extends between vertebral members V1, V2 and together these structures define a vertebral joint. The system 20 may also be used if all or a portion of disc D has been removed and replaced with a fusion or motion preserving implant. In the example systems 20 shown, the devices 10 are positioned at a posterior side of the spine, on opposite sides of the spinous processes S. In alternative embodiments, spinal devices 10 may be attached to a spine at other locations, including lateral and anterior locations. Spinal devices 10 may also be attached at various sections of the spine, including the base of the skull and to vertebrae in the cervical, thoracic, lumbar, and sacral regions. Thus, the illustration in FIG. 1 is provided merely as a representative example of one application of a spinal stabilization system 20.

In the exemplary system 20, the spinal devices 10 are secured to vertebral members V1, V2 by connector assemblies 12 comprising a pedicle screw 14 and a retaining cap 16. The outer surface of spinal device 10 is grasped, clamped, or otherwise secured between the pedicle screw 14 and retaining cap 16. In alternative embodiments, the connector assemblies may allow sliding motion of the spinal device. Other mechanisms for securing spinal devices 10 to vertebral members V1, V2 include hooks, cables, and other such devices. Further, examples of other types of retaining hardware include threaded caps, screws, and pins. Thus, the exemplary assemblies 20 shown in FIG. 1 are merely representative of one type of attachment mechanism.

For the present discussion, an exemplary elongated connecting element is described as a rod assembly, but other elements and structures may be used, such as a plate, hollow cylinder, blocks, discs, etc., without departing from the spirit and scope of the invention. The invention is not limited to a rod and is limited only by the claims appended hereto. Moreover, if a rod is used, it is not limited to a circular cross section, but may have an oval, rectangular, hexagonal, or any other regular or irregular cross section shape without departing from the spirit and scope of the invention. The rod may be curved, non-curved, or capable of being curved, depending on the circumstances of each application.

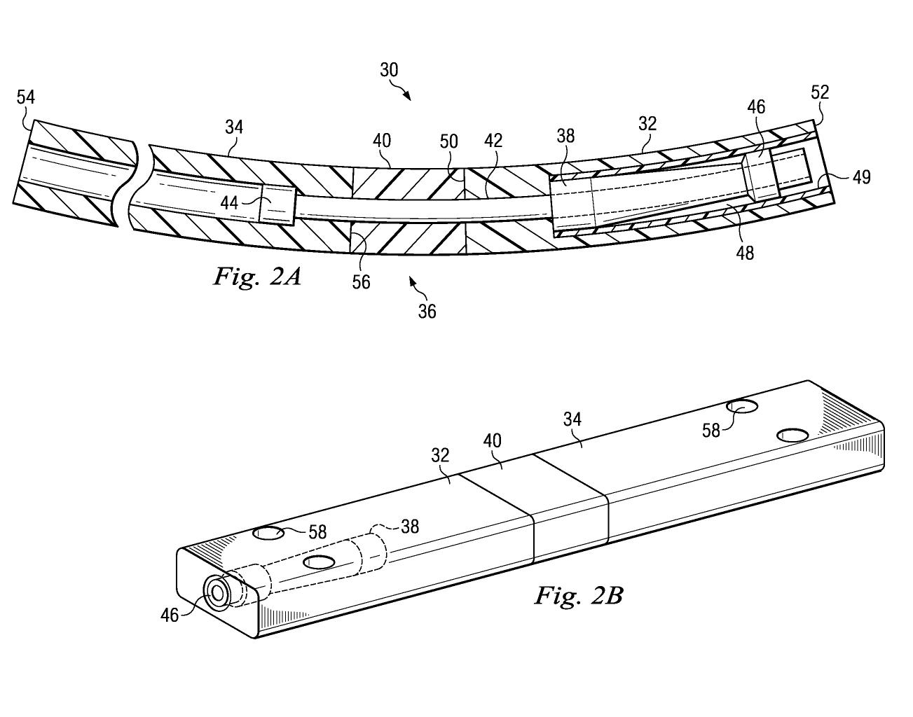

FIG. 2A illustrates a rod assembly 30 which may be used as the spinal device 10 of system 20. The rod assembly 30 has a first section 32, a second section 34, and a dampening assembly 36. As better illustrated in FIG. 2, the dampening assembly 36 includes a first elastomer 38, a second elastomer 40, and a connector 54. The connector 42 has an anchor end 44 and a piston end 46. As shown in FIG. 1, the anchor end 44 of the connector 42 is anchored in the second section 34 and the piston end 46 is disposed within the first section 32. The piston end 46 may be connectable to the connector 42 for ease of assembly (See FIG. 4). For example the piston end 46 may be threaded or fused to the connector 42. In an alternative embodiment, the piston end may be integrally formed with the connector 42.

A cavity 48 is defined by the first section 32 and the first elastomer, or flexion dampening elastomer, 38 is disposed within the cavity 48. The cavity may be provided with a sleeve 49. In one embodiment, the cavity is substantially cylindrical, but the cavity may be in the shape of a rectangular prism, a hexagonal prism, conical or frustoconical shape, or any other shape.

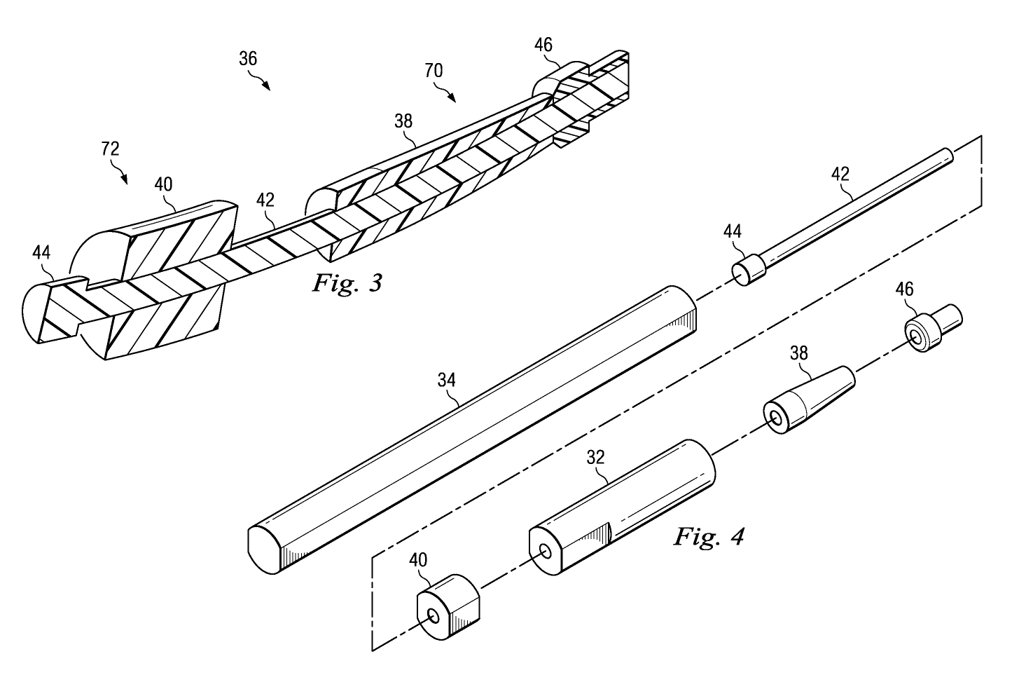

The second elastomer, or extension dampening elastomer, 40 is located between the first section 32 and the second section 34. The connector 42 extends through the second elastomer 40, through the first elastomer 38, and terminates in the piston end 46. The piston end 46 is outside of the first elastomer 38, but still within the cavity 48. As illustrated in FIGS. 2A and 3, the piston end 46 abuts the first elastomer 38; however, the piston end 46 may also be spaced away from the first elastomer 38 or embedded within first elastomer 38 without departing from the spirit and scope of the invention. It is within the spirit and scope of the invention for the first elastomer to be the extension dampening elastomer and the second elastomer to be the flexion dampening elastomer.

The first section 32 has a first end 50 and a second end 52 and the second section 34 has a first end 54 and a second end 56. As illustrated in the embodiment in FIG. 2A, the second elastomer 40 abuts the second section second end 56 and the first section first end 50. The first elastomer 38 and the second elastomer 40 may not abut each other, but rather, may be separated by a portion of the first section 32. In other words, the cavity 48 may not extend to the first section first end 50. Alternatively, the cavity 48 could extend such that first elastomer 38 and second elastomer 40 abut each other.

The first section second end 52 may be open to the cavity 48, as illustrated in FIG. 2A, or closed without departing from the spirit and scope of the invention.

FIG. 4 illustrates an exploded view of embodiment of the rod assembly 30 showing first section 32, second section 34, first elastomer 38, second elastomer 40, connector 42, anchor end 44, and piston end 46. One method of assembly is to insert anchor end 44 into the second section 34 and crimp or otherwise secure the anchor end 44 in the second section 34. The securing, anchoring, or other mechanical retaining of anchor end 44 may be done in any conventional manner.

The second elastomer 40 is then placed onto the connector 42 and the first section 32 is placed onto connector 42 after the second elastomer 40. First elastomer 38 is placed onto the connector 42 and into the cavity 48 within the first section 32. Then the piston end 46 is secured in a conventional manner, such as by crimping, to the connector 42. Either first elastomer 38 or second elastomer 40 can be pre-loaded by compression or tension, if desired, during the assembly process.

Other methods of assembly will be apparent to one of ordinary skill in the art without undue experimentation depending on the specific elements selected for assembly.

FIG. 2B illustrates another embodiment of the present invention in which the first section 32 and the second section 34 are configured as plates, with holes 58 provided for attachment to vertebra, such as via pedicle screws.

The first section 32 and the second section 34 can be constructed of any suitable material, preferably a biocompatible material. Examples of material that can be used include cobalt-chromium alloys, titanium alloys, nickel titanium alloys, and/or stainless steel alloys, any member of the polyaryletherketone (PAEK) family such as polyetheretherketone (PEEK), carbon-reinforced PEEK, or polyetherketoneketone (PEKK); polysulfone; polyetherimide; polyimide; ultra-high molecular weight polyethylene (UHMWPE); and/or cross-linked UHMWPE. Any combination of these materials may also be suitable. For example, a suitable material may include a layer of carbon fiber reinforced PEEK inside an otherwise uniform PEEK material. The first and second sections may have a common base material, however the first section may have a different modulus of elasticity than the second elastomer through the use of molding techniques or material reinforcements.

The first elastomer 38 and the second elastomer 40 can be constructed of any suitable material, preferably a biocompatible material. The first elastomer and the second elastomer are, for example, flexible and resilient or elastic to permit motion of the spinal motion segment with which they are associated while providing a desired stabilization effect. The first elastomer 38 and the second elastomer 40 can be constructed such that one or both has a gradual or otherwise variable stiffness. Examples of material that can be used include any suitable biocompatible elastomer or polymer biomaterial, such as surgical latex, chloroprene, MIT's “biorubber” (glycerol and sebacic acid), polyethylene, polyester, polyurethane, urethane, polypropylene, silicone, or hydrogel, and combinations thereof. The first elastomer and the second elastomer can also be constructed in the form of a spring or any other shape exhibiting elastomeric properties from any suitable material. Examples of such material include cobalt-chromium alloys, titanium alloys, nickel titanium alloys, and/or stainless steel alloys.

In some embodiments, the first elastomer 38, the second elastomer 40 or both are constructed at least partially of a resorbable material. The first elastomer 38 or second elastomer 40 will then gradually resorb into the body, allowing for gradually increasing movement over time. In some embodiments, the first elastomer 38 or second elastomer 40 has one or more components 38A, 40A, as discussed in more detail below, that are resorbable to selectively modify the amount of movement increase over time. See FIGS. 8 and 9. The resorbable component or components 38A, 40A are arranged in parallel or series with one or more non-resorbable, or permanent, components to provide additional adaptability of the stiffness, resiliency and elasticity of the first elastomer 38 or second elastomer 40 based on the circumstances of use.

The first elastomer 38 and the second elastomer 40 may have the same construction or may have different construction. The first elastomer and the second elastomer may have the same or different characteristics, such as shape, size, length, stiffness, elasticity, resiliency, etc. Either or both elastomers may be multi-durometer or have gradual or discrete changes in the stiffness, resiliency, or elasticity over the length, width, or diameter of the elastomer.

The connector 42 may be flexible or inflexible, elastic, inelastic, or semi-elastic and of any suitable form, such as a tether, suture, wire, band, cord, cable, rope, or a solid or hollow rod, for example. The connector 42 can be single strand, multiple strands, braided, or combinations thereof and constructed of any suitable material, preferably a biocompatible material. Examples of possible materials include but are not limited to woven or non-woven polymers, such as polyester, polyethylene, or any member of the polyaryletherketone (PAEK) family such as polyetheretherketone (PEEK), carbon-reinforced PEEK, or polyetherketoneketone (PEKK), polysulfone; polyetherimide, polyimide, ultra-high molecular weight polyethylene (UHMWPE), and/or cross-linked UHMWPE; superelastic metals, such as nitinol; shape memory alloy, such as nickel titanium; resorbable synthetic materials, such as suture material, metals, such as stainless steel and titanium; synthetic materials, allograft material; and bioelastomer material.

The sleeve 49 can be constructed of any suitable material, preferably a biocompatible material. Examples of material that can be used include cobalt-chromium alloys, titanium alloys, nickel titanium alloys, and/or stainless steel alloys, any member of the polyaryletherketone (PAEK) family such as polyetheretherketone (PEEK), carbon-reinforced PEEK, or polyetherketoneketone (PEKK); polysulfone; polyetherimide; polyimide; ultra-high molecular weight polyethylene (UHMWPE); and/or cross-linked UHMWPE.

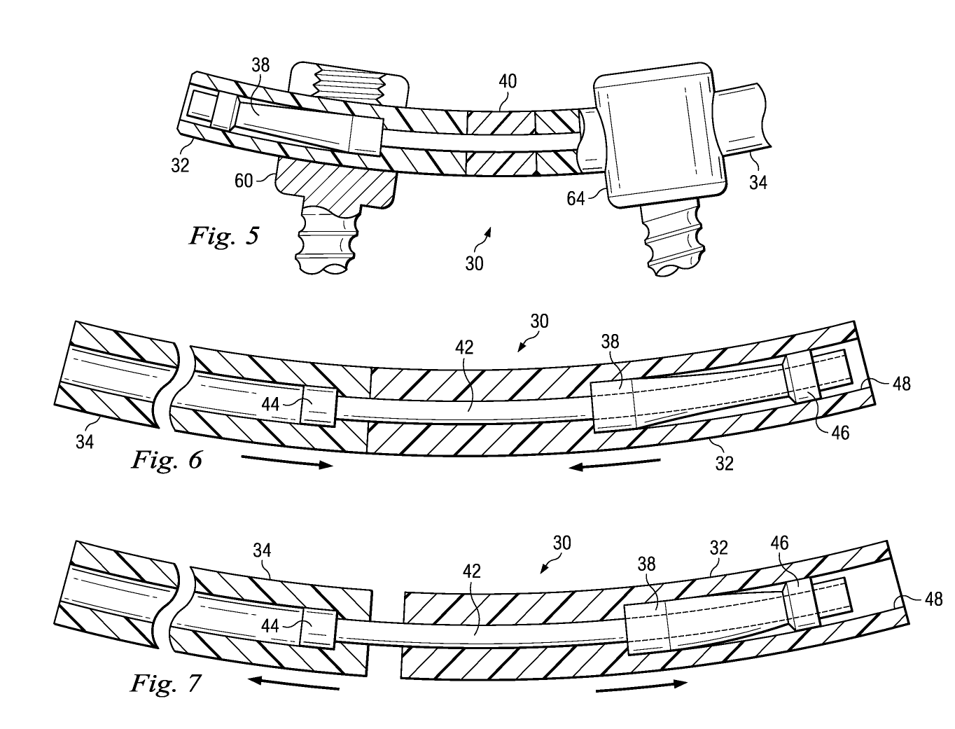

FIG. 5 illustrates a spinal stabilization system in accordance with one embodiment of the present invention in which a first bone anchor assembly 60 is attachable to a first vertebra (not shown) and a second bone anchor assembly 64 is attachable to a second vertebra (not shown). The first section 32 of the elongated connecting element, or rod assembly, 30 is connected to the first bone anchor assembly 60 and the second section 34 of the elongated connecting element, or rod assembly, 30 is connected to the second bone anchor assembly 64. The first bone anchor assembly 60 and the second bone anchor assembly 64 are attachable, for example, to the pedicles of adjacent vertebra. The attachment may be to non-adjacent vertebra, or other than to the pedicles of the vertebra, without departing from the spirit and scope of the invention. For convenience of description, reference will be made to adjacent vertebra and attachment to the pedicles of the vertebra.

The bone anchor assemblies 60, 64 are any conventional bone anchor assemblies capable of or designed for attachment to vertebrae in any conventional manner. The elongated connecting element 30 may be used with new bone anchor assemblies 60, 64 that are packaged or included with the elongated connecting element 30 as a system or the elongated connecting element 30 may be used for revision surgery with bone anchor assemblies 60, 64 that were implanted into vertebrae at a separate time.

The elongated connecting element first section 32 and the second section 34 may be securable directly to the first and second bone anchor assemblies 60, 64, or there may be another element present to facilitate connection to the bone anchor assemblies 60, 64. For example, the first section 32 or the second section 34 may be fitted with a collar made of any suitable material and the collar then is secured directly to the bone anchor assembly. The first section 32 and the second section 34 are directly or indirectly secured to the bone anchor assemblies 60, 64 in any conventional manner.

The first vertebra and the second vertebra may be adjacent vertebra or non-adjacent vertebra. In either case, the attachment of the elongated connecting element to the bone anchor assemblies will provide dynamic stabilization of the spine in the area of the first vertebra and the second vertebra to which the bone anchor assemblies are attached. This dynamic stabilization allows some motion of the spine between the first and second vertebra, but also dampens that motion.

If the patient bends backward, that exerts force on the connecting element/rod assembly 30 to move the first section 32 and the second section 34 toward each other to compress the rod assembly. This includes movement of the first section 32 toward the second section 34, the movement of second section 34 toward the first section 32, or both.

If the connecting element/rod assembly 30 were a rigid, inflexible rod, then the first and second vertebra would be unable to move relative to each other. However, the second elastomer 40 described herein is flexible and at least partially elastic and enables motion of the first section 32 and the second section 34 toward each other by compressing the second elastomer 40. The first section 32 and the second section 34 each press the second elastomer 40 from substantially opposite directions, compressing the second elastomer 40 and enabling movement of the first section 32 and the second section 34 toward each other. This, in turn, enables the vertebra to which the first section 32 and the second section 34 are attached to move relative to each other.

The material of construction of the second elastomer 40 may be selected to provide the desired amount of allowed motion of the first section 32 and the second section 34 toward each other, depending on the elasticity and quantity of the material chosen as well as the specific configuration of the shape of the second elastomer 40. Thus, the motion of the first section 32 and the second section 34 toward each other may be selectively limited, or dampened.

If the patient bends forward, that exerts force on the connecting element/rod assembly 30 to move the first section 32 and the second section 34 away from each other to expand the rod assembly. This includes movement of the first section 32 away from the second section 34, the movement of second section 34 away from the first section 32, or both.

If the connecting element/rod assembly 30 were a rigid, inflexible rod, then the first and second vertebra would be unable to move relative to each other. However, the first elastomer 38 described herein is flexible and at least partially elastic and enables motion of the first section 32 and the second section 34 away from each other by compressing the first elastomer 38. It is surprising that the dampening of the flexion movement, and of movement of the first section 32 and the second section 34 away from each other, is accomplished by compression of the first elastomer 38 instead of by stretching an elastomer.

As can be seen in FIG. 2A, the anchor end 44 of the connector 42 is anchored in the second section 34. The connector 42 extends through the first elastomer 38 and terminates in the piston end 46 which is disposed at the opposite end of the first elastomer 38 from the anchor end 44. When the forces are applied to first section 32 and second section 34 to move apart relative to each other, the piston end 46 exerts a compression force on the first elastomer 38, which is confined within the cavity 48. This enables the first section 32 and the second section 34 to move away from each other, but also limits, or dampens, the movement. This, in turn, enables the vertebra to which the first section 32 and the second section 34 are attached to move relative to each other.

The material of construction of the first elastomer 38 may be selected to provide the desired amount of allowed motion of the first section 32 and the second section 34 away from each other, depending on the elasticity and quantity of the material chosen as well as the specific configuration of the shape of the first elastomer 38. Thus, the motion of the first section 32 and the second section 34 away from each other may be selectively limited, or dampened.

The material and details of construction of the first elastomer 38 and the second elastomer 40 are selected so that, for example, one elastomer is stiffer, or has a different durometer, than the other elastomer. Thus, the resistance of the each of the elastomers to pressure may be different to allow for more flexion movement than extension movement or more extension movement than flexion movement. This enables selected and customized dampening of flexion and extension. “Flexion” is the forward bending of the spine. “Extension” is the backward bending of the spine.

As one example, several different possible first elastomers 38 and second elastomers 40 having different stiffness, shape, etc., properties are provided to the surgeon, such as in a kit, to allow the surgeon to select a first elastomer 38 and a second elastomer 40 from a variety of components. Then the specific elongated connecting element can be assembled, such as described above, prior to surgery. As another example, the surgeon can assemble, or have assembled, an elongated connecting element in which the first elastomer 38 is assembled from several components. The surgeon selects, for example, a first component having a first stiffness and a second component having a second stiffness and those are threaded onto the connector 42 to form a single first elastomer 38. Likewise, a second elastomer 40 may be assembled from one or more separate components having different properties. The components can be elastomers or non-elastomers, resorbable or non-resorbable, such that the resulting first elastomer 18 and second elastomer 40 are elastomers, as described above.

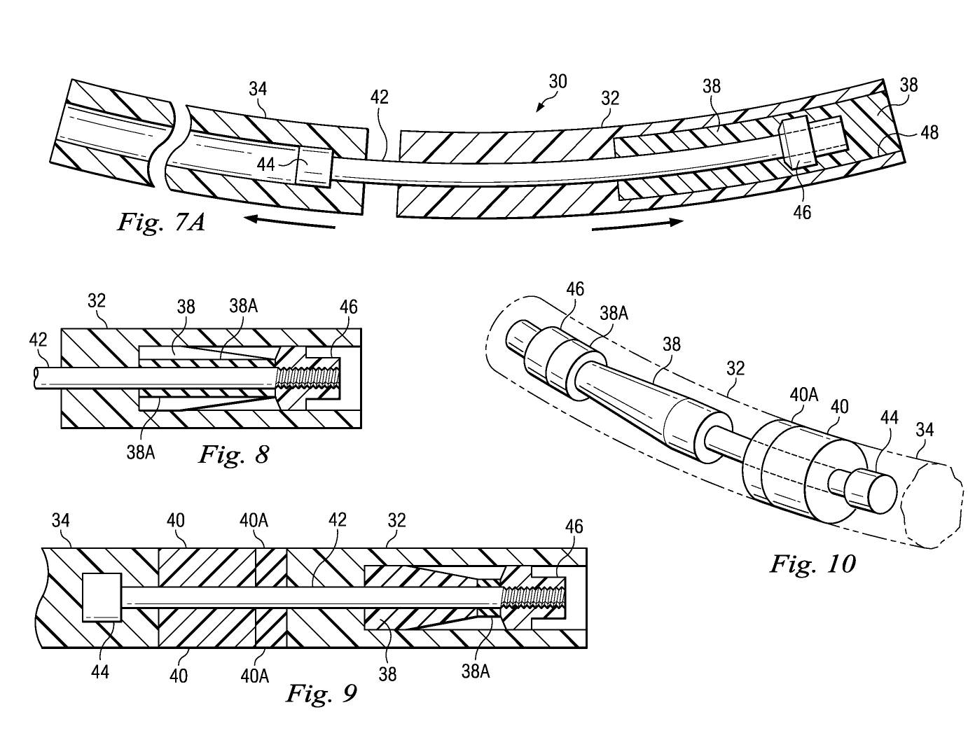

In some situations, there will only be a need for an elongated connecting element to enable and limit, or dampen, the movement of the first section and second section away from each other, such as during spinal flexion. FIGS. 6 and 7 illustrate an embodiment of the connecting element 30 present invention in which the second elastomer 40 is not present. This embodiment includes the first section 32, the second section 34, the first elastomer, or flexion dampening elastomer 38, the connector 42, the anchor end 44, the piston end 46, and the cavity 48. In this embodiment, extension of the spine will not result in dampened movement of the first section 32 and the second section 34 toward each other, because there is no compressible second elastomer 40 disposed between the first section 32 and the second section 34. But upon flexion of the spine, the first section 32 and the second section 34 will move away from each other, as illustrated in FIG. 7, by the same mechanism described above.

Alternatively, as shown in FIG. 7a, the elongated connecting element 30 includes a first section 32, a second section 34, and a first elastomer 38 disposed within first section 32, as described above, that enables and limits, or dampens, both flexion and extension of the vertebra. In this embodiment, for example, connector 42 is not flexible and piston end 46 is disposed within the first elastomer 38 in the cavity 48. The first elastomer 38 is bounded at both ends by the cavity 48, or otherwise constrained within the first section 32, such that exertion of force in any direction will result in compression of the first elastomer 38. Thus, the non-flexible connector 42 will exert a compression force on the first elastomer 38 via the piston end 46 regardless of whether the force applied is to move first section 32 and second section 34 away from each other or toward each other.

As indicated above, the specific shape of first elastomer 38 and second elastomer 40 may be selected without departing from the spirit and scope of the invention. For convenience of description and illustration, the shape of the first elastomer 38 and the second elastomer 40 has been described and illustrated above as substantially cylindrical. As an alternate example, FIG. 10 illustrates an embodiment of the present invention in which first elastomer 38 has a substantially frustoconical or conical shape. FIG. 10 also illustrates resorbable components 38A and 40A, which may or may not be present, as discussed above. Other shapes for first elastomer 38, second elastomer 40, and resorbable components 38A and 40A are also contemplated and within the scope of the invention.

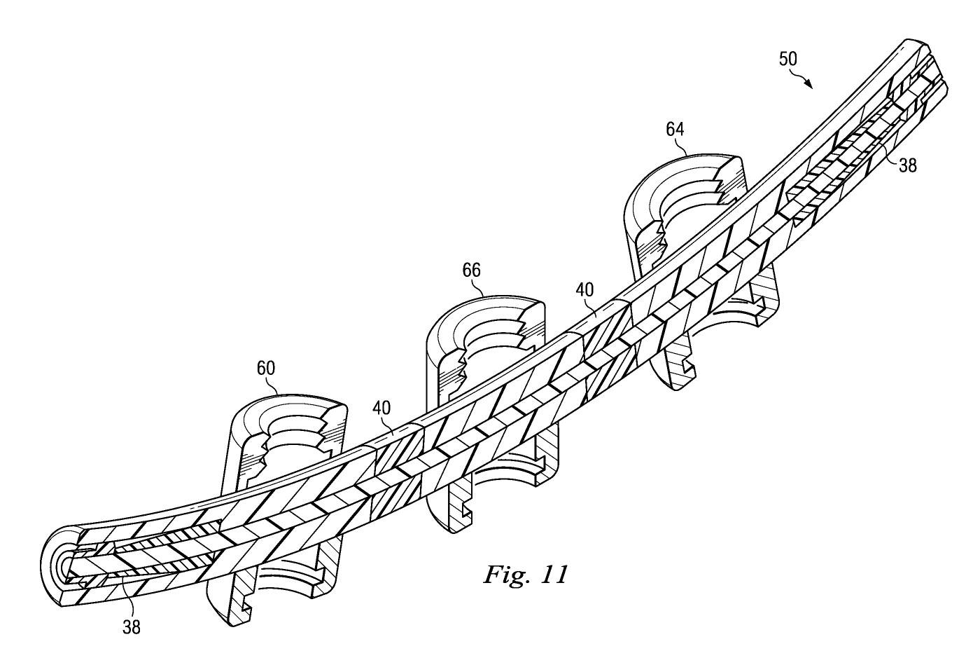

FIG. 11 illustrates another embodiment in which first bone anchor assembly 60, second bone anchor assembly 64, and a third bone anchor assembly 66 are included. This arrangement is used, for example, when the rod assembly 30 is to be attached across at least three vertebra with each bone anchor assembly attachable to a different vertebra. FIG. 11 also illustrates a rod assembly 30 in which there are two first elastomers 38 and two second elastomers 40. In the illustrated embodiment, the first elastomers 38 are substantially frustoconical, but may be of any shape.

As described, the elongated connecting element, or rod assembly, 30 has at least two regions. A first region, generally associated with first section 32 is configured to enable and to limit, or dampen, the expansion of the element, such as when the first vertebra and the second vertebra are in flexion. This has the result of enabling and limiting flexion of the vertebra to which the connecting element is attached.

In still another alternative example, the first region includes the anchored connector and the first section having a cavity similar to cavity 48, but with a viscous fluid or gel disposed in the cavity and sealed in by the piston end. As another example, the cavity is substantially sealed with a fluid therein, and the piston end is provided with a valve arrangement to control the flow of fluid for dampening. As yet another example, the connector may also have some elasticity and the interaction between the selectively elastic connector and the selectively elastic first elastomer brings about the desired dampening effect. A further example includes the first elastomer being a spring element. In yet another example, the first section does not define a cavity, but the first elastomer is integral with the first section. Each of these examples, and their equivalents, are included as the first elastomer, flexion dampening elastomer, rebound elastomer, or rebound element.

As described, the elongated connecting element, or rod assembly, 30 has also a second region, generally between first section 32 and second section 34, which is configured to enable and to limit, or dampen, the compression of the element, such as when the first vertebra and the second vertebra are in extension. This has the result of enabling and limiting extension of the vertebra to which the connecting element is attached.

As described above, in one embodiment, the second region includes the second elastomer between the first section and the second section. As another example, the second region includes a spring element between first section and second section. As a further example, the second region includes an integral portion of the connecting element that has a different modulus of elasticity (Young's modulus) than the first section and the second section, enabling some compression of the second region. Other means for enabling and limiting, or dampening, extension of the connected vertebra are within the spirit and scope of the invention

In some embodiments, the first region and the second region are separate and distinct from each other, although they may be connected and communicate with each other.

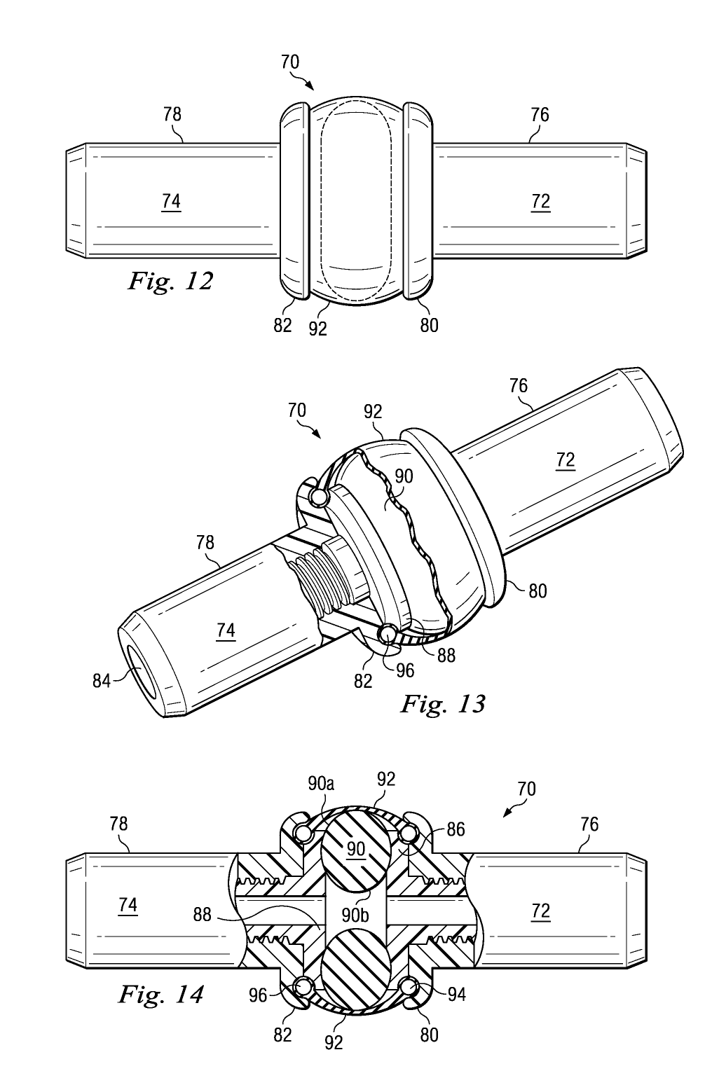

Referring now to FIGS. 12-14, in this embodiment a spinal device 70 may be used as the spinal device 10 of system 20. The spinal device 70 includes end anchors 72, 74 which include extension portions 76, 78, respectively. In this embodiment, extension portions 76, 78 may be generally cylindrical, but other shapes including curves or non-circular cross-sections may also be suitable. The end anchors 72, 74 also include endplates 80, 82, respectively. A hollow passage 84 may end through end anchor 74, and a corresponding hollow passage may pass through the end anchor 76. Anchor plates 86, 88 may be threadedly engaged with the hollow passages of the end anchors 74, 76, respectively. For example, the anchor plate 88 may be threaded into the hollow passage 84 until the anchor plate abuts the endplate 82.

A bumper 90, which in this embodiment has a generally toroidal shape with an outer radial surface 90a and an inner radial surface 90b. The bumper 90 extends between the anchor plates 86, 88. In alternative embodiments, the bumper may be solid (i.e., lacking a center aperture), dome-shaped, frusto-conical, or other shapes that may be apparent to one skilled in the art. A sheath 92 may circumferentially surround the bumper 90 and be connected between the endplates 80, 82 by fasteners 94, 96, respectively. In this embodiment, the fasteners 94, 96 may be, for example, wires recessed into a circumferential groove on the endplates 80, 82.

The end anchors 72, 74 can be constructed of any suitable material, preferably a biocompatible material. Examples of materials that can be used include cobalt-chromium alloys, titanium alloys, nickel titanium alloys, and/or stainless steel alloys, any member of the polyaryletherketone (PAEK) family such as polyetheretherketone (PEEK), carbon-reinforced PEEK, or polyetherketoneketone (PEKK); polysulfone; polyetherimide; polyimide; ultra-high molecular weight polyethylene (UHMWPE); and/or cross-linked UHMWPE. Suitable ceramic materials may include carbon-based materials or alumina-based materials.

The bumper 90 may be formed of any suitable biocompatible elastomer or polymer biomaterial, such as surgical latex, chloroprene, MIT's “biorubber” (glycerol and sebacic acid), polyethylene, polyester, polyurethane, urethane, polypropylene, silicone, or hydrogel, and combinations thereof. The bumper may be formed of a single material having a single durometer measurement or modulus of elasticity. Alternatively the bumper may have regions of differing hardness. For example a core section of the bumper may be formed of a material having a higher modulus of elasticity than an outer portion. Such a construction would allow greater initial compression but eventually limit further compression. Alternatively, the bumper may have differences in durometer at different lateral locations along the bumper to permit, for example flexion-extension, but limit lateral bending. Multiple durometers would allow flexion and extension stiffness to be designed into the device.

The sheath 92 may be biocompatible and flexible materials such as a segmented polyurethane, BIOSPAN-S (aromatic polyetherurethaneurea with surface modified end groups from Polymer Technology Group), CHRONOFLEX AR/LT (aromatic polycarbonate polyurethane with low-tack properties from CardioTech International), CHRONOTHANE B (aromatic polyether polyurethane from CardioTech International), CARBOTHANE PC (aliphatic polycarbonate polyurethane from Thermedics). The sheath may be permeable or impermeable, and in some embodiments may be a woven textile.

The spinal device 70 may be installed at a vertebral joint using, for example, connector assemblies 12 to attach the end anchors 72, 74 to the vertebrae V1, V2. The device 70 may be used to control flexion and extension motion and to resist shear loads. During a flexion motion, the end anchors 72, 74 may transmit a tensile load to the sheath 92 that will compress the outer radial surface of the bumper 90 radially inward. The sheath 92 may further serve as a tether to limit excessive flexion motion. During extension, the end anchors 72, 74 may transmit a compressive load to the anchor plates 86, 88 which may apply a compressive force to the bumper, causing the outer radial surface 90a of the bumper to extend radially outward. The flexible nature of the sheath allows this radial movement of the bumper.

In an alternative embodiment, the anchor plates may be omitted and the endplates 80, 82 may directly engage the bumper. In another alternative embodiment, the anchor plates or endplates may be angled to control the transmission of shear forces.

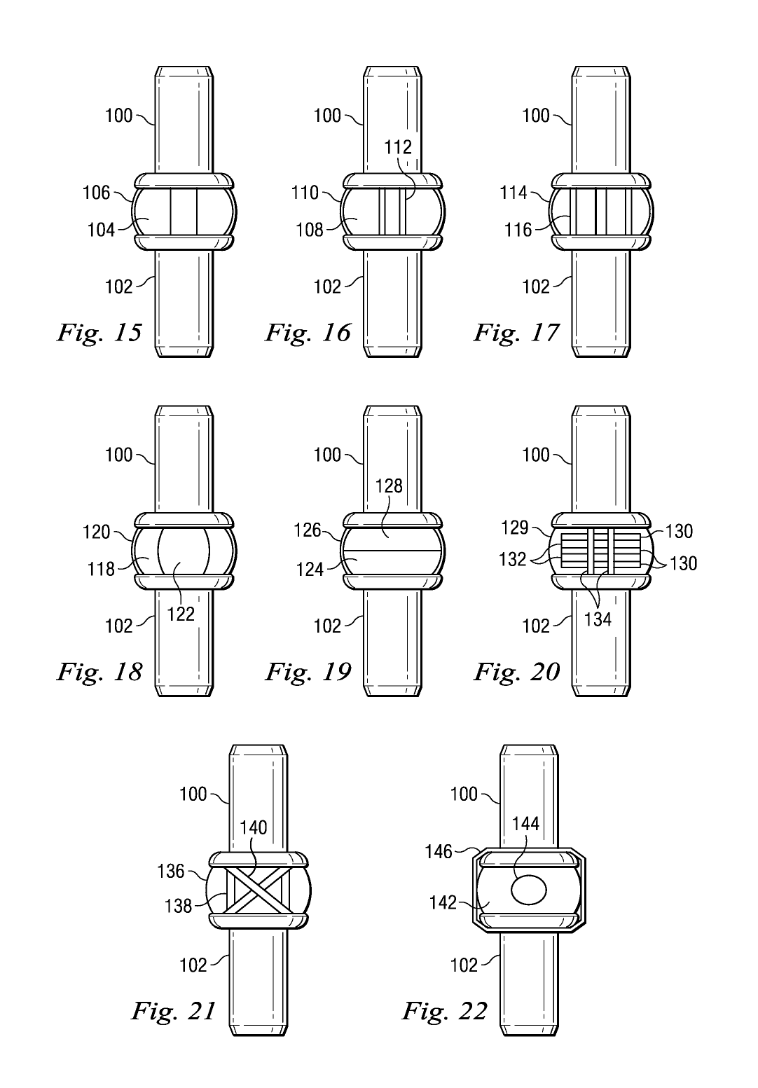

FIGS. 15-22 depict partial cross-sectional views of spinal devices that may be used as the spinal device 10 of system 20. The spinal devices each include end anchors 100, 102 which may be formed of metal, ceramic, or polymers such as those described above for end anchors 72, 74. In FIG. 15, a bumper 104 may have a generally toroidal or cylindrical shape and may be formed of a material such as those described above for bumper 90. A sheath 106 may connect between the end anchors 100, 102, encapsulating the bumper. The sheath 106 may be formed of a flexible material such as those described above for sheath 92.

In FIG. 16, a bumper 108 may have a generally toroidal or cylindrical shape and may be formed of a material such as those described above for bumper 90. A sheath 110 may connect between the end anchors 100, 102 and surround the outer surface of the bumper 108. The sheath 110 may be formed of a flexible material such as those described above for sheath 92. A second sheath 112 may extend between the end anchors 100, 102 and surround the inner surface of the bumper 108. Under flexion loading, both sheaths 110 and 112 will transmit a tensile load to compress or squeeze the bumper 108 radially inward from both the outer and inner surfaces. The sheaths 110, 112 may further serve to limit excessive flexion motion.

In FIG. 17, a bumper 116 may have a generally toroidal or cylindrical shape and may be formed of a material such as those described above for bumper 90. A flexible material 116 may extend through the bumper 116 and connect between the end anchors 100, 102. The material 116 may be formed of a flexible material such as those described above for sheath 92. The material 116 may serve as a tether to limit excessive flexion motion and provide reinforcement responsive to shear loading.

In FIG. 18, an outer bumper 118 may have a generally toroidal or cylindrical shape and may be formed of a material such as those described above for bumper 90. A sheath 120 may connect between the end anchors 100, 102, encapsulating the bumper. The sheath 120 may be formed of a flexible material such as those described above for sheath 92. An inner bumper 122 may extend inside the outer bumper 118 and between the end anchors 100, 102. The inner bumper 122 may be formed of an elastomeric material such as those described for bumper 90. In this embodiment, the inner bumper 122 may have a harder or softer modulus or elasticity or durometer measurement than the outer bumper.

In FIG. 19, a lower bumper 124 may have a generally inverted dome shape and may be formed of a material such as those described above for bumper 90. An upper bumper 128 may be positioned on top of the lower bumper 124. The upper bumper 122 may also be formed of an elastomeric material such as those described for bumper 90. In this embodiment, the upper bumper 122 may have a harder or softer modulus or elasticity or durometer measurement than the lower bumper. The upper and lower bumpers 124, 128 may be affixed or integrally molded with one another or alternatively, may be allowed to float with respect to one another. A sheath 126 may connect between the end anchors 100, 102, encapsulating the bumpers 124, 128. The sheath 126 may be formed of a flexible material such as those described above for sheath 92.

In FIG. 20, a bumper 129 comprises alternating layers of two elastomeric material 130 and 132, each having different moduli of elasticity or durometer measurements. The bumper 129 is positioned between the end anchors 100, 102. In this embodiment, a flexible material 134 may extend through the layered bumper 129 and connect between the end anchors 100, 102. The material 134 may be formed of a flexible material such as those described above for sheath 92. The material 134 may serve as a tether to limit excessive flexion motion and provide reinforcement responsive to shear loading.

In FIG. 21, a bumper 136 may have a generally disc shape and may be formed of a material such as those described above for bumper 90. A flexible material 138 may extend linearly through the bumper 136 and connect between the end anchors 100, 102. The material 138 may be formed of a flexible material such as those described above for sheath 92. The material 138 may serve to limit excessive flexion motion and provide reinforcement responsive to shear loading. Flexible tethers 140 may extend through the bumper at an angle and connect between the end anchors 100, 102. The angled tethers 140 may assist with torsion resistance as well as shear resistance and flexion resistance.

In FIG. 22, an outer bumper 142 may have a generally disc shape and may be formed of a material such as those described above for bumper 90. An inner bumper 144 may be encapsulated within the outer bumper 142. The inner bumper 144 may also be formed of an elastomeric material such as those described for bumper 90. In this embodiment, the inner bumper 144 may have a harder or softer modulus or elasticity or durometer measurement than the outer bumper 142. A sheath 146 may connect between the end anchors 100, 102, encapsulating the bumper and at least a portion of the end anchors 100, 102. The sheath 146 may be formed of a flexible material such as those described above for sheath 92. The sheath 146 may provide resistance to excessive flexion motion and may also serve to contain wear debris.

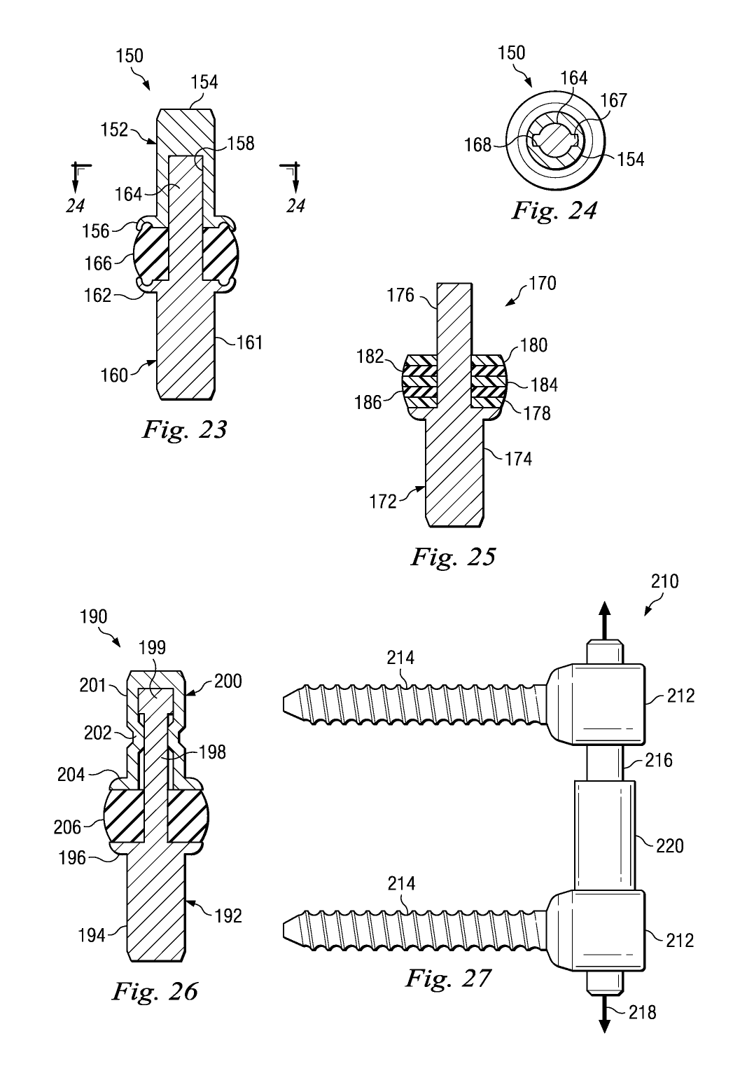

Referring now to FIGS. 23 and 24, in this embodiment a spinal device 150 may be used as the spinal device 10 of system 20. The spinal device 150 includes an end anchor 152 which includes an extension portion 154 and an endplate 156. The end anchor 152 further includes an internal bore 158 extending through the endplate 156 and at least partially through the extension portion 154. In alternative embodiments, the internal bore may pass entirely through the extension portion, resulting in the extension portion having a tubular configuration. The spinal device 150 further includes an end anchor 160 which includes an extension portion 161 and an endplate 162. The end anchor 160 further includes a rod portion 164 extending from the endplate 162 in an opposite direction from the extension portion 161. The spinal device 150 further includes a bumper 166. In this embodiment, extension portions 154, 161 may be generally cylindrical, but other shapes including curves or non-circular cross-sections may also be suitable. In this embodiment, the extension portion 161, the endplate 162, and the rod portion 164 may be integrally formed. In alternative embodiments, the sections may be modular. For example, the extension portion could be threadably connected to the rod portion.

The end anchors 152, 160 can be constructed of any suitable material, preferably a biocompatible material. Examples of material that can be used include cobalt-chromium alloys, titanium alloys, nickel titanium alloys, and/or stainless steel alloys, any member of the polyaryletherketone (PAEK) family such as polyetheretherketone (PEEK), carbon-reinforced PEEK, or polyetherketoneketone (PEKK); polysulfone; polyetherimide; polyimide; ultra-high molecular weight polyethylene (UHMWPE); and/or cross-linked UHMWPE. Suitable ceramic materials may include carbon-based materials or alumina-based materials.

The bumper 166 may be formed of any suitable biocompatible elastomer or polymer biomaterial, such as surgical latex, chloroprene, MIT's “biorubber” (glycerol and sebacic acid), polyethylene, polyester, polyurethane, urethane, polypropylene, silicone, or hydrogel, and combinations thereof. The bumper may be formed of a single material having a single durometer measurement or modulus of elasticity. An example of a suitable durometer hardness may be between 50A and 75D. Alternatively the bumper may have regions of differing hardness. For example a core section of the bumper may be formed of a material having a higher modulus of elasticity than an outer portion. Such a construction would allow greater initial compression but eventually limit further compression. Alternatively, the bumper may have differences in durometer at different lateral locations along the bumper to permit, for example flexion-extension, but limit lateral bending. Multiple durometers would allow flexion and extension stiffness to be designed into the device. The bumper 166 may also be provided in different heights to allow a surgeon to select an appropriate distraction height.

The spinal device 150 may be assembled by sliding the bumper 166 over the rod portion 164 such that rod portion extends through the bumper and the bumper contacts the endplate 162. The rod portion 164 may then be inserted into the internal bore 158 of the end anchor 152. The internal bore 158 may be long enough that the endplate 156 contacts the bumper 166. As assembled, the end anchor 152 may be allowed to slide or float on the rod portion 164. The bumper 166 may also be allowed to slide or float on the rod portion 164. Alternatively, the bumper may be affixed to either the endplate 162 or the endplate 156. As shown in FIG. 24, to prevent the rod portion 164 from rotating within the internal bore 158, the rod portion may include an outwardly extending key 167 configured to slide within a key channel 168 of the internal bore 158. In alternative embodiments, the channel may be formed in the rod portion and the outwardly extending key formed on the internal bore. Other interdigitating features which may prevent rotation of the rod portion relative to the internal bore are also suitable.

The end anchors 152, 160 may be locked to the vertebrae V1, V2 with connecting assemblies 12. As implanted the device 150 may permit both flexion and extension motion. In extension, the bumper 166 may serve to block excessive extension. Depending upon the hardness of the bumper 166, the bumper may provide either a hard stop or a dampened soft stop.

Referring now to FIG. 25, in this embodiment a spinal device 170 may be used as the spinal device 10 of system 20. The spinal device 170 includes an end anchor 172 which includes an extension portion 174, a rod portion 176, and an endplate 178. In this embodiment, the extension portion 174, the endplate 178, and the rod portion 176 may be integrally formed. In alternative embodiments, the sections may be modular. For example, the extension portion could be threadably connected to the extension portion. The end anchor 172 may be formed of a suitable biocompatible material such as those described for end anchor 160.

The device 170 further includes a series of bumpers 180, 182, 184, 186. The bumpers 180-186 may be formed of different materials or have different hardnesses. Fewer or more layers of bumpers may be used. The bumpers 180-186 may be formed of a suitable biocompatible material such as those described above for bumper 166.

The device 170 may be installed by locking the end anchor extension portion 174 to the vertebra V2 with a connecting assembly 12, which may be a fixed connection. The rod portion 176 may be slidably attached to vertebra V1 with a sliding connector that allows the rod portion 176 to slide within the sliding connector relative to the vertebra V1. In one alternative embodiment, the rod portion 176 may include a stop at an end opposite the endplate 178 to prevent the sliding connector from decoupling from the rod portion. As implanted, the device 170 may permit both flexion and extension motion. In extension, the series of bumpers 180-186 may serve to block excessive extension. Depending upon the hardness of the bumpers, the bumper may provide either a hard stop or a dampened soft stop.

Referring now to FIG. 26, in this embodiment a spinal device 190 may be used as the spinal device 10 of system 20. The spinal device 190 includes an end anchor 192 which includes an extension portion 194, an endplate 196, and a rod portion 198. A stop portion 199 may be located on the rod portion 198 at the end opposite the endplate 196. The spinal device 190 further includes an end anchor 200 which includes an extension portion 201, an endplate 204, and a crimped section 202. The end anchor 200 further includes an internal bore 203 extending through the endplate 204 and at least partially through the extension portion 201. In alternative embodiments, the internal bore may pass entirely through the extension portion, resulting in the extension portion having a tubular configuration. The spinal device 190 further includes a bumper 206. In this embodiment, the extension portion 194, the endplate 196, and the rod portion 198 may be integrally formed. In alternative embodiments, the sections may be modular. For example, the extension portion could be threadably connected to the rod portion.

The end anchor 192 may be formed of a suitable biocompatible material such as those described for end anchor 160. The bumper 206 may be formed of a suitable biocompatible material such as those described above for bumper 166.

The spinal device 190 may be assembled by sliding the bumper 206 over the rod portion 198 such that rod portion extends through the bumper and the bumper contacts the endplate 196. The rod portion 164 may then be inserted into the internal bore 203 of the end anchor 200. The internal bore 203 may be long enough that the endplate 204 contacts the bumper 206. The stop 199 may be forced past the crimped section 202 so that the stop 199 becomes trapped within the internal bore 203 by the crimped section 202. Alternatively, the crimped section may be formed after the stop 199 has been inserted fully into the internal bore 203. Creating the crimped section after the stop has been inserted would eliminate the need to temporarily deform the end anchor 200 to force the stop past the crimped section.

As assembled, the end anchor 200 may be allowed to slide or float on the rod portion 198. The bumper 206 may also be allowed to slide or float on the rod portion 198. Alternatively, the bumper may be affixed to either the endplate 196 or the endplate 204. To prevent the rod portion from rotating within the internal bore, the rod portion may include an outwardly extending key such as described above in FIG. 24. The end anchors 192, 200 may be locked to the vertebrae V1, V2 with connecting assemblies 12. As implanted the device 190 may permit both flexion and extension motion. In extension, the bumper 206 may serve to block excessive extension. Depending upon the hardness of the bumper 166, the bumper may provide either a hard stop or a dampened soft stop. In this embodiment, the crimped portion 202 may prevent the end anchor portion 192 from dislocating from the end anchor portion 200.

Referring now to FIG. 27, in this embodiment a spinal device 210 may be used as the spinal device 10 of system 20. The spinal device 210 may be attached to vertebrae V1, V2 with connecting assemblies which in this embodiment include head portions 212 and bone screw portions 214. The spinal device 210 includes a rod portion 216 which extends along a longitudinal axis 218. The spinal device 210 further includes a sleeve portion 220 which extends over the rod portion 216 and shares the central longitudinal axis 218.

The rod portion 216 may be formed of a flexible biocompatible material including, for example, a material of the polyaryletherketone (PAEK) family such as polyetheretherketone (PEEK), carbon-reinforced PEEK, or polyetherketoneketone (PEKK); polysulfone; polyetherimide; polyimide; ultra-high molecular weight polyethylene (UHMWPE); and/or cross-linked UHMWPE. Flexible ceramic or metals may also be suitable. The sleeve portion may be formed of any of the materials described for the rod portion 216 or alternatively may be formed of a more resilient material such as the materials described above for bumper 166.

As assembled and installed the spinal device 210 may permit both flexion and extension. In extension, the sleeve portion 220 may serve to limit extension by preventing further bending of the rod portion 216 when the upper head portion 212 of the connecting assembly contacts the sleeve portion. As can be readily understood, a shorter sleeve portion may permit more extension than would a longer sleeve portion.

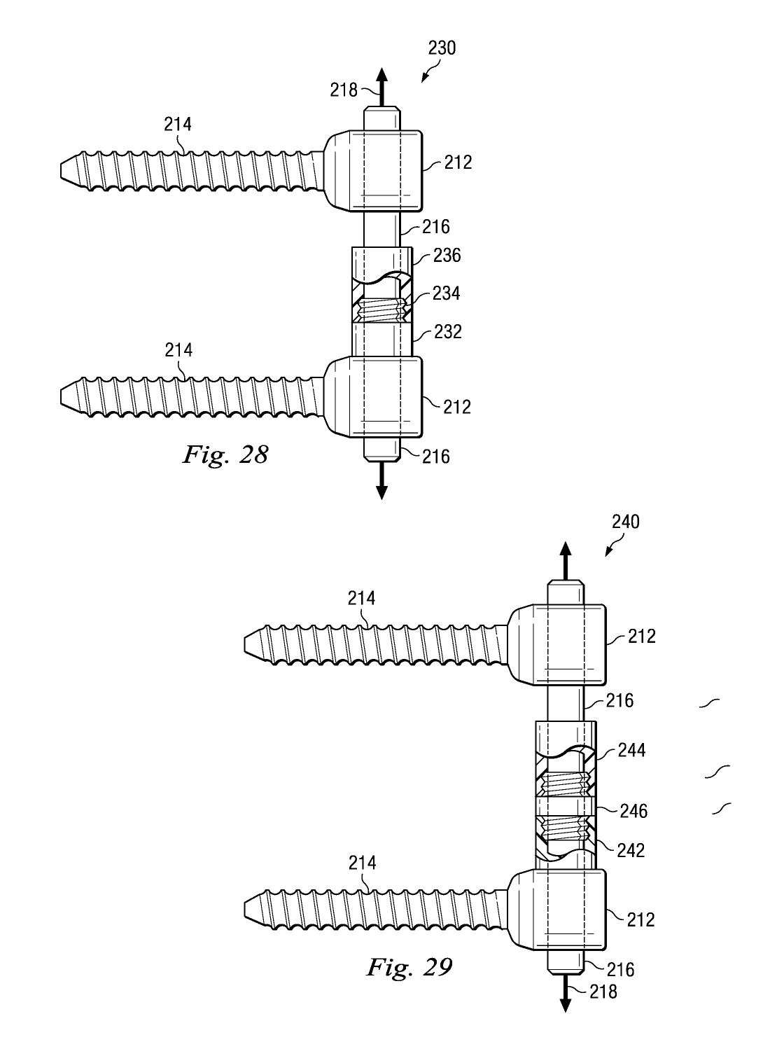

As shown in FIGS. 28 and 29, the sleeve height may be adjustable in situ in situations where it may be difficult to determine pre- or intra-operatively the proper distraction height of the sleeve. In FIG. 28, a spinal device 230 may be used as the spinal device 10 of system 20. The spinal device 230 may be attached to vertebrae V1, V2 with connecting assemblies which in this embodiment include head portions 212 and bone screw portions 214. The spinal device 230 includes the rod portion 216 which extends along the longitudinal axis 218. In this embodiment, the spinal device 230 further includes a sleeve portion comprising sleeve section 232 and a sleeve section 236 both of which extend over the rod portion 216. Sleeve section 232 may include an outer threaded section 234 which mates with an inner threaded section of the sleeve section 236. The overall height of the combined sleeve portion may be controlled by adjusting the threaded connection between the section 232 and the section 236.

In an alternative embodiment, one of the sections 232, 236 could be connected to a drive system which may include a receiver and a motor. The receiver may receive remote signals and control the action of the motor to turn one of the sections 232, 236 to increase or decrease the overall height of the sleeve portion. The drive system may be enclosed in a pacemaker style housing so that the height of the sleeve portion may be adjusted after the surgical implantation has occurred. Such a post-operative adjustment could be used to remove a segmental kyphosis if the height was too great upon insertion. Alternately, the distraction height may be increased post-operatively if, for example, subluxation of the facets occurred during extension.

In still another alternative embodiment, the overall height of the combined sleeve portion and/or the distance between the head portions 212 may be adjusted with a pump system. In this embodiment, the threaded connection may be omitted and replaced with a fluid pump to increase or decrease the overall height. To increase the overall height of the sleeve portions, a catheter may be inserted into the patient and into a connection on the spinal device. A syringe may be connected to the catheter. To increase the overall height, the syringe may deliver fluid to the pump via the catheter. To decrease the overall height, the syringe may remove fluid from the pump.

In still another alternative embodiment, the overall height of the combined sleeve portion and/or the distance between the head portions 212 may be adjusted by manipulating a mechanical driver such as a screw or a jack. For example, a cannula may be inserted into the patient to access a screw head of the screw. A screwdriver may be passed through the cannula to turn the screw head and thereby increase or decrease the overall height.

In FIG. 29, a spinal device 240 may be used as the spinal device 10 of system 20. The spinal device 240 may be attached to vertebrae V1, V2 with connecting assemblies which in this embodiment include head portions 212 and bone screw portions 214. The spinal device 240 includes the rod portion 216 which extends along the longitudinal axis 218. In this embodiment, the spinal device 240 further includes a sleeve portion comprising sleeve section 242, a sleeve section 244, and an intermediate sleeve section 246, all of which extend over the rod portion 216. Intermediate sleeve section 246 may include outer threads which mate with inner threaded sections of the sleeve sections 242, 244. For example, sleeve section 244 may have left hand threads and the sleeve section 242 may have right hand threads. In this example, the intermediate section 246 may have a single thread pattern. The overall height of the combined sleeve portion may be controlled by adjusting the threaded connection between the sections 242, 244 and the intermediate section 246.

In an alternative embodiment, the intermediate section 246 or one or both of the sections 242, 244 could be connected to a drive system which may include a receiver and a motor. The receiver may receive remote signals and control the action of the motor to turn one of the sections to increase or decrease the overall height of the sleeve portion.

These embodiments in which the height of the spinal device may be increased may be particularly useful for pediatric applications in which the patient's spine grows naturally and the spinal device should be adjusted to track the growth of the patient.

### Specific Embodiments

A elongated connecting element for use in a spinal stabilization system comprises, a first section; a second section; a first elastomer disposed within the first section; and a second elastomer disposed between the first section and the second section. One of the first elastomer and the second elastomer resists movement of the first section and the second section toward each other and the other of the first elastomer and the second elastomer resists movement of the first section and the second section away from each other.

The connecting element further comprising a connector anchored in the second section, extending completely through the second elastomer, and extending at least partially through the first elastomer.

The connector comprises polymer braid, weave, or monofilament.

The first section defines a cavity in which the first elastomer is disposed, the cavity comprising a first end and a second end.

The first section comprises a liner disposed within the cavity.

The first elastomer and the second elastomer are not adjacent to each other.

The first section and the second section comprise the same or different material selected from the group consisting of cobalt-chromium alloy, titanium alloy, nickel titanium alloy, and/or stainless steel alloy, and any member of the polyaryletherketone family.

The first elastomer and the second elastomer comprise the same or different material selected from the group consisting of polyethylene, polyester, polyurethane, urethane, polypropylene, silicone, or hydrogel, and combinations thereof.

The first elastomer comprises a different material than the second elastomer.

The first elastomer has a different resiliency than the second elastomer.

The first elastomer or the second elastomer comprises a plurality of elastomeric components.

At least one of the first elastomer and the second elastomer comprises a resorbable component.

A rod assembly for use in a spinal stabilization system comprises a first region configured to enable dampened expansion of the rod assembly upon flexion of the spine. The rod assembly further includes a separate second region configured to enable dampened compression of the rod assembly upon extension of the spine.

The first region comprises a first elastomer disposed within a first section of the rod assembly.

The second region comprises a second elastomer disposed between a first section and a second section of the rod assembly.

A system for stabilization of a spine, comprises a first bone anchor assembly capable of attachment to a first vertebra; a second bone anchor assembly capable of attachment to a second vertebra; and an elongated connecting element. The elongated connecting element comprises a first section for attachment to the first bone anchor assembly; a second section for attachment to the second bone anchor assembly; a first region configured to enable dampened expansion of the connecting element upon flexion of the spine; and a separate second region configured to enable dampened compression of the connecting element upon extension of the spine.

The first region comprises a first elastomer disposed within the first section of the connecting element.

The second region comprises a second elastomer disposed between the first section and the second section of the connecting element.

The system further comprises a connector anchored in the second section, extending through the second elastomer and at least part of the first elastomer, terminating in an end in communication with the first elastomer.

A rod assembly for attachment to vertebrae in a spinal stabilization system, the rod comprising first means to enable and limit flexion of the vertebrae, and second means to enable and limit extension of the vertebra, wherein the first means is different from the second means.

The first means comprises a first elastomer disposed within a cavity defined within the rod assembly.

The second means comprises a second elastomer disposed between a first section and a second section of the rod assembly.

A rod assembly for use in a spinal stabilization system comprises a first section; a second section; and a rebound element disposed within the first section. The rebound element enables and dampens movement of the first section and the second section away from each other.

The rod assembly further comprises a connector anchored in the second section and extending at least partially through the rebound element.

The connector is in communication with the rebound element such that when the first section and the second section are moved away from each other, the connector exerts force on the rebound element and the resistance of the rebound element to the exerted force dampens the movement of the first section and the second section away from each other.

The rebound element enables and dampens movement of the first section and the second section toward each other.

A rod assembly for use in a spinal stabilization system comprises a first section defining a cavity; a second section; a connector anchored in the second section and connecting the first section to the second section; and a flexion dampening elastomer disposed within the cavity. The connector communicates with the flexion dampening elastomer to enable and dampen movement of the first section and the second section away from each other.

The rod assembly further comprises a dampening elastomer disposed between the first section and the second section, the dampening elastomer selected to enable and limit movement of the first section toward the second section. The connector extends through the dampening elastomer.

While the present invention has been illustrated by the above description of embodiments, and while the embodiments have been described in some detail, it is not the intention of the applicant to restrict or in any way limit the scope of the invention to such detail. Additional advantages and modifications will readily appear to those skilled in the art. Therefore, the invention in its broader aspects is not limited to the specific details, representative apparatus and methods, and illustrative examples shown and described. Accordingly, departures may be made from such details without departing from the spirit or scope of the applicant's general or inventive concept. It is understood that all spatial references, such as “horizontal,” “vertical,”“top,” “upper,” “lower,” “bottom,” “left,” and “right,” are for illustrative purposes only and can be varied within the scope of the disclosure. In the claims, means-plus-function clauses are intended to cover the structures described herein as performing the recited function and not only structural equivalents, but also equivalent structures.

What is claimed is:

1. A elongated connecting element for use in a spinal stabilization system, the connecting element comprising:
a first section;
a second section;
a first elastomer disposed within the first section; and
a second elastomer disposed between the first section and the second section wherein one of the first elastomer and the second elastomer resists movement of the first section and the second section toward each other and the other of the first elastomer and the second elastomer resists movement of the first section and the second section away from each other.

2. The element of claim 1, further comprising a connector anchored in the second section, extending completely through the second elastomer, and extending at least partially through the first elastomer.

3. The element of claim 2, wherein the connector comprises a braid, weave, or monofilament.

4. The element of claim 1, wherein the first section defines a cavity in which the first elastomer is disposed, the cavity comprising a first end and a second end.

5. The element of claim 4, wherein the first section comprises a liner disposed within the cavity.

6. The element of claim 1, wherein the first elastomer and the second elastomer are not adjacent to each other.

7. The element of claim 1, wherein the first section and the second section comprise the same or different material selected from the group consisting of cobalt-chromium alloy, titanium alloy, nickel titanium alloy, and/or stainless steel alloy, and any member of the polyaryletherketone family.

8. The element of claim 1, wherein the first elastomer and the second elastomer comprise the same or different material selected from the group consisting of polyethylene, polyester, polyurethane, urethane, polypropylene, silicone, or hydrogel, and combinations thereof.

9. The element of claim 1, wherein the first elastomer and second elastomer comprise a common base material and wherein the first elastomer has a different modulus of elasticity than the second elastomer.

10. The element of claim 1, wherein the first elastomer has a different resiliency than the second elastomer.

11. The element of claim 1, wherein the first elastomer or the second elastomer comprises a plurality of elastomeric components.

12. The element of claim 1, wherein at least one of the first elastomer and the second elastomer comprises a resorbable component.

13. An elongated connecting element for use in a spinal stabilization system, the connecting element comprising:
first and second end anchors and
an elastomeric bumper engaged between the first and second end anchors, the elastomeric bumper including an outer radial surface,
wherein movement of the first and second end anchors toward each other presses the outer radial surface of the bumper radially outward and movement of the first and second end anchors away from each other presses the outer radial surface radially inward.

14. The elongated connecting element of claim 13 further comprising a first sheath extending between the first and second end anchors and around the outer radial surface of the bumper.

15. The elongated connecting element of claim 13 wherein the elastomeric bumper has a toroidal shape.

16. The elongated connecting element of claim 14 further comprising a second sheath extending between the first and second end anchors and around an inner radial surface of the bumper.

17. The elongated connecting element of claim 13 wherein the bumper comprises first and second portions, each portion having a different durometer hardness.

18. An elongated connecting element for use in a spinal stabilization system, the connecting element comprising:
a first end anchor comprising a first elongated cylindrical section and an internal bore extending at least partially through the elongated cylindrical section;
a second anchor comprising a second elongated cylindrical section and a rod portion extending away from the second elongated cylindrical section; and
a bumper between the first and second elongated cylindrical sections,
wherein the rod portion is sized to extend through the bumper and into the internal bore of the first end anchor.

19. The elongated connecting element of claim 18 wherein the rod portion includes an outward projection to resist rotation of the rod portion within the internal bore.

20. The elongated connecting element of claim 18 wherein the internal bore includes a crimped section to resist dislocation of the rod portion from the internal bore.

21. A spinal stabilization system comprising:
first and second bone connecting assemblies;
a flexible elongated connecting element extending between the first and second bone connecting assemblies; and
an adjustable sleeve extending over at least a portion of the connecting element.

22. The spinal stabilization system of claim 21 wherein the adjustable sleeve comprises a first portion threadedly connectable to a second portion.

23. The spinal stabilization system of claim 22 wherein the adjustable sleeve comprises a third portion threadedly connectable to the second portion.

24. A method of stabilizing a spinal joint comprising:
inserting a first connecting assembly into a first vertebra;
inserting a second connecting assembly into a second vertebra;
extending an elongated connecting element between the first and second connecting assemblies;
extending an adjustable sleeve over the elongated connecting element, the adjustable sleeve including a first sleeve portion movably connected to the second sleeve portion; and
actuating a drive system to adjust a height of the adjustable sleeve by moving the first sleeve portion with respect to the second sleeve portion.

25. The method of claim 24 wherein the step of actuating the drive system includes post-operatively and remotely actuating the drive system.
